(19) United States
(12) Patent Application Publication
(10) Pub. No.: US 2018/0346940 A1
(43) Pub. Date: Dec. 6, 2018

(54) COMPOSITIONS AND METHODS FOR THE PRODUCTION OF HYDROCARBONS, HYDROGEN AND CARBON MONOXIDE USING ENGINEERED AZOTOBACTER STRAINS

(71) Applicant: The Regents of the University of California, Oakland, CA (US)

(72) Inventors: Markus W. RIBBE, Irvine, CA (US); Yilin HU, Chicago, IL (US); Johannes REBELEIN, Irvine, CA (US); Chi LEE, Irvine, CA (US)

(21) Appl. No.: 15/779,268

(22) PCT Filed: Nov. 21, 2016
(86) PCT No.: PCT/US2016/063123

§ 371 Date: May 25, 2018

Related U.S. Application Data
(60) Provisional application No. 62/260,434, filed on Nov. 27, 2015.

(51) Int. Cl. C12P 7/14; C12P 5/02; C12N 15/113; C12N 9/00; C12R 1/065

(52) U.S. Cl. CPC C12P 7/14; C12P 5/02; C12R 1/065; C12N 9/00; C12N 15/113

(57) ABSTRACT

In alternative embodiments, provided are genetically or recombinantly engineered nitrogen-fixing, nitrogenase-expressing bacteria capable of enzymatically synthesizing hydrocarbons and generating hydrogen and carbon monoxide, and for carbon dioxide and/or carbon monoxide recycling, and compositions (e.g., bioreactors and devices) for using them, and methods for making and using them. In alternative embodiments, the genetically or recombinantly engineered nitrogen-fixing, nitrogenase expressing bacteria used to practice embodiments provided herein include nitrogen-fixing diazotrophs such as nitrogen-fixing bacteria of the family Pseudomonadaceae, or the genus Azotobacter, including Azotobacter vinelandii, for the whole cell synthesis of hydrocarbons and generating hydrogen and carbon monoxide, and for the recycling of carbon dioxide and/or carbon monoxide.

### PRIORITY CLAIM AND REFERENCE TO RELATED APPLICATION

The application claims priority under 35 U.S.C. §119 and all applicable statutes and treaties from prior U.S. provisional application Ser. No. 62/260,434, which was filed Nov. 27, 2015. The aforementioned application is expressly incorporated herein by reference its entirety and for all purposes.

### TECHNICAL FIELD

This invention generally relates to bioreactors, biofuels and compositions and processes for improving and saving the environment. In alternative embodiments, provided are genetically or recombinantly engineered nitrogen-fixing, nitrogenase-expressing bacteria capable of enzymatically synthesizing hydrocarbons and generating hydrogen and carbon monoxide, and for carbon dioxide and/or carbon monoxide recycling, and compositions (e.g., bioreactors and devices) for using them, and methods for making and using them. In alternative embodiments, the genetically or recombinantly engineered nitrogen-fixing, nitrogenase expressing bacteria used to practice embodiments provided herein include nitrogen-fixing diazotrophs such as nitrogen-fixing bacteria of the family Pseudomonadaceae, or the genus Azotobacter, including Azotobacter vinelandii, for the whole cell synthesis of hydrocarbons and generating hydrogen and carbon monoxide, and for the recycling of carbon dioxide and/or carbon monoxide.

### BACKGROUND OF THE INVENTION

Hydrocarbons such as propane, butane, and other alkanes and alkenes are in widespread use, both as fuels and as the precursors for many vital and necessary chemical compounds such as plastics, detergents, pharmaceuticals, etc. Currently the primary sources of these hydrocarbons are fossil fuels, such as natural gas, from which they can be isolated. Such natural sources are, however, necessarily available in limited supply, and retrieval and processing can have undesirable environmental impacts. In addition, the availability and pricing of such fossil fuels is greatly impacted by unpredictable political and social events.

Chemoautotrophic microorganisms which are able to utilize inorganic carbon have been grown in a bioreactor using carbon dioxide (CO₂) as a carbon source. Growth of these bacteria provides a biomass that may then be dried and harvested for useful components, for instance lipids and fats can be extracted from dried biomass using solvents and after additional processing may subsequently be used as fuels. Reactor designs are, however, complex in order to accommodate the environmental requirements for chemoautotrophic bacteria. In addition, while this approach does provide reduction of inorganic carbon under relatively mild conditions the resulting product is a highly complex mixture of biomolecules that requires extensive processing in order to isolate useful compounds.

There is a need for systems and methods that can provide reduction of inorganic carbon, such as CO and CO₂, to generate hydrocarbons under mild conditions.

### SUMMARY OF THE INVENTION

In alternative embodiments, provided are methods or systems, including whole cell methods or systems, for enzymatically synthesizing a hydrocarbon, a carbon monoxide, a hydrogen or a hydrocarbon, carbon monoxide and hydrogen, comprising:

(a) providing a nitrogen-fixing bacteria of the family Pseudomonadaceae, optionally of the genus Azotobacter, optionally an Azotobacter vinelandii,

wherein the bacteria are genetically or recombinantly engineered to lack, substantially lack or have decreased molybdenum transporter activity, optionally by deletion of a molybdenum transporter gene or by inhibition of molybdenum transporter expression, optionally by DNA or RNA targeting and cleavage or modification by a CRISPR-Cas9 system,

and optionally the bacteria are genetically or recombinantly engineered to: express an exogenous nitrogenase, optionally a vanadium nitrogenase; express more endogenous nitrogenase, optionally vanadium nitrogenase; and/or have increased nitrogenase, e.g., vanadium nitrogenase, activity,

(b) providing a culture environment or a container for the nitrogen-fixing bacteria of (a), wherein the culture environment or container comprises:

a culture fluid or media for growing or culturing the nitrogen-fixing bacteria,

a liquid input to the culture fluid or media for inputting liquid nutrient and a liquid outlet for outputting liquid waste; and

a gas or air input for inputting gas and a gas or culture atmosphere outlet for outputting or releasing gas;

(c) providing a gas and a hydrocarbon separation device operatively linked to the culture environment or container, wherein the gas input and the gas outlet of the culture environment or container are operably connected to the gas and the hydrocarbon separation device, wherein gas output of the culture environment or container passes through the gas outlet to the gas and hydrocarbon separation device, which separates out (optionally substantially removes) hydrogen and/or hydrocarbons from the gas output of the culture environment or container, and the gas output of the culture environment or container from which hydrogen and/or hydrocarbons are at least substantially removed are returned to the culture environment or container through the gas input for the culture environment or container;

and optionally carbon monoxide (CO) is also separated out (optionally substantially removed) from the gas output of the culture environment or container by the gas and hydrocarbon separation device and recycled back to the culture environment or container through the gas input for the culture environment or container,

and optionally the CO-comprising gas output of the gas and hydrocarbon separation device is mixed with additional CO before inputting to the culture environment or container, and optionally sufficient additional CO is added to the CO-comprising gas output of the gas and hydrocarbon separation device such that a relatively stable amount of CO is recycled into or passed into the culture environment or container,

and optionally the amount of CO recycled or passed back into the culture environment or container is in the form of a CO gas-air mixture comprising between about 5% to 35% CO, between about 12% to 15% CO, or between about 10% to 17% CO, or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or 31% CO,

and optionally the amount of CO recycled or passed back into the culture environment or container is regulated or maintained by a value and a valve actuator or equivalent, wherein optionally the valve actuator or equivalent is operably linked to a CO detection device in the culture environment or container and an operating system such that the amount of CO passed into the culture environment or container by the value and value actuator maintains the culture environment or container gas environment at between about 5% to 35% CO, between about 12% to 15% CO, or between about 10% to 17% CO, or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or 31% CO; and

(d) culturing or incubating the nitrogen-fixing bacteria in the culture environment or container under conditions wherein the nitrogen-fixing bacteria generate hydrocarbons, CO and/or hydrogen, and inputting to the culture fluid or media a liquid nutrient, and outputting from the culture fluid or media a liquid waste, and inputting to the culture fluid or media a gas or air mixture comprising CO and air, and outputting gas from the culture fluid or media to the gas and hydrocarbon separation device.

In alternative embodiments, provided are methods or systems wherein the suitable culture fluid or media comprises a Burke's minimal medium or equivalent supplemented with 2 mM ammonium or equivalent and 30 μM Na₃VO₄ or equivalent.

In alternative embodiments, provided are methods or systems wherein the gas and hydrocarbon separation device comprises more than one device or apparatus, or comprises a gas chromatograph (GC) or a GC-TCD (a GC with a thermal conductivity detector), or a GC-FID (a GC with flame ionization detector) optionally with methanizer, or equivalents.

In alternative embodiments, provided are methods or systems wherein the hydrocarbons produced or generated by the nitrogen-fixing bacteria and separated by the gas and hydrocarbon separation device comprise propane (C₃H₈), ethane (C₂H₆), ethylene (C₂H₄) or any C2 to C10 hydrocarbon, optionally comprising alkanes and alkenes.

In alternative embodiments, provided are methods or systems wherein the hydrocarbons, hydrogen and/or CO produced or generated by the nitrogen-fixing bacteria and separated by the gas and hydrocarbon separation device are separated and separately saved or harvested, and optionally all or part of the CO is recycled back to the culture environment or a container.

In alternative embodiments, provided are methods or systems wherein the hydrogen and CO produced or generated by the nitrogen-fixing bacteria and separated by the gas and hydrocarbon separation device are harvested and packaged together to produce a syngas.

In alternative embodiments, provided are methods or systems wherein the hydrogen produced or generated by the nitrogen-fixing bacteria and separated by the gas and hydrocarbon separation device is recycled back to the culture environment or a container, optionally for hydrogenation of hydrocarbons generated by the nitrogen-fixing bacteria.

In alternative embodiments, provided are methods or systems wherein the A. vinelandii comprises an A. vinelandii strain YM68A.

In alternative embodiments, provided are methods or systems comprising a method, process or system as illustrated in FIG. 1, FIG. 2, FIG. 3 or FIG. 5.

In alternative embodiments, provided are methods or systems, including whole cell methods and systems, for enzymatically converting a carbon dioxide to a carbon monoxide and/or a hydrocarbon, comprising:

(a) providing a nitrogen-fixing bacteria of the family Pseudomonadaceae, optionally of the genus Azotobacter, optionally an Azotobacter vinelandii,

wherein the bacteria are genetically or recombinantly engineered to: lack, substantially lack or have decreased activity in one or both subunits of the molybdenum-iron (MoFe) or vanadium-iron (VFe) component of nitrogenase (NifD and NifK for MoFe component, or VnfD and VnfK for VFe component, respectively),

and either: permit the expression of an iron protein component of a nitrogenase (NifH for Mo-nitrogenase, VnfH for V-nitrogenase), augment expression of an iron protein component of a nitrogenase, and/or genetically or recombinantly engineer an enzymatic activity comprising an iron protein component of a nitrogenase;

(b) providing a culture environment or a container for the nitrogen-fixing bacteria of (a), wherein the culture environment or container comprises:

a culture fluid or media for growing or culturing the nitrogen-fixing bacteria,

a liquid input to the culture fluid or media for inputting liquid nutrient and a liquid outlet for outputting liquid waste; and

a gas or air input for inputting gas and a gas or culture atmosphere outlet for outputting or releasing gas;

(c) culturing or incubating the nitrogen-fixing bacteria in the culture environment or container under conditions wherein the nitrogen-fixing bacteria generate hydrocarbons and/or CO, and inputting to the culture fluid or media a liquid nutrient, and outputting from the culture fluid or media a liquid waste, and inputting to the culture fluid or media a gas or air mixture comprising carbon dioxide, and outputting gas from the culture fluid or media.

In alternative embodiments, provided are methods or systems that further comprise operably linking with any method or system as provided herein, wherein the carbon monoxide or hydrocarbon generated by any method or system as provided herein is inputted or recycled into the culture environment or a container of any method or system as provided herein.

In alternative embodiments, provided are methods or systems wherein the gas or air mixture comprising carbon dioxide inputted to the culture fluid or media a liquid nutrient comprises an air or a gas mixture comprising between about 10% and 90% carbon dioxide.

In alternative embodiments, provided are genetically or recombinantly engineered nitrogen-fixing bacteria (a bacterium) of the family Pseudomonadaceae, optionally of the genus Azotobacter, optionally an Azotobacter vinelandii, wherein the bacteria (or bacterium) are genetically or recombinantly engineered to: lack, substantially lack or have decreased activity in one or both subunits of the molybdenum-iron (MoFe) or vanadium-iron (VFe) component of nitrogenase (NifD and NifK for MoFe component, or VnfD and VnfK for VFe component, respectively), and either: permit the expression of an iron protein component of a nitrogenase (NifH for Mo-nitrogenase, VnfH for V-nitrogenase), augment expression of an iron protein component of a nitrogenase, and/or genetically or recombinantly engineer an enzymatic activity comprising an iron protein component of a nitrogenase.

In alternative embodiments, provided are products of manufacture such as devices, bioreactors, reactors and fermenters comprising genetically or recombinantly engineered nitrogen-fixing bacteria described or provided herein.

In alternative embodiments, provided are products of manufacture such as devices, bioreactors, reactors and fermenters comprising a method or system as provided herein.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

Figures are described in detail herein.

FIG. 1 schematically illustrates an exemplary system, such as a bioreactor or a device, as provided herein to practice an exemplary method for the whole cell (e.g., bacterial) production of hydrogen, hydrocarbons and/or carbon monoxide (CO), as discussed in detail, below.

FIG. 2 schematically illustrates an exemplary system used to practice exemplary methods as provided herein, e.g., for the whole cell (e.g., bacterial) production of hydrogen, hydrocarbons and/or carbon monoxide (CO), as discussed in detail, below.

FIG. 3 schematically illustrates an exemplary system used to practice exemplary methods as provided herein, e.g., for the whole cell (e.g., bacterial) production of hydrogen, hydrocarbons and/or carbon monoxide (CO), as discussed in detail, below.

FIG. 4 graphically illustrates data from an exemplary method as provided herein, which shows the mol H2/mol nitrogenase as a function of 100% air, 15% CO and 85% air and 30% CO and 70% air (with V-nitrogenase as the left bar of each pair of bars, and V-nitrogenase as the right of each pair of bars), as discussed in detail, below.

FIG. 5 schematically illustrates an exemplary system used to practice exemplary methods as provided herein, e.g., for the whole cell (e.g., bacterial) production of hydrogen, hydrocarbons and/or carbon monoxide (CO), as discussed in detail, below.

FIG. 6 graphically illustrates data from an exemplary method as provided herein, showing that the CO formation from CO₂ by genetically modified strains of A. vinelandii expressing NifH of Mo-nitrogenase (most left-hand bar of each four bar set, or the black bars) and VnfH of V-nitrogenase (the second from right of each set of 4 bars, or the green bars), as discussed in detail, below.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, provided are systems, reactors (e.g., bioreactors), devices and processes and methods for whole cell production of hydrocarbons, hydrogen and carbon monoxide, and for the recycling of carbon dioxide and/or carbon monoxide. In alternative embodiments, provided are genetically or recombinantly engineered nitrogen-fixing, nitrogenase-expressing bacteria capable of enzymatically synthesizing hydrocarbons and generating hydrogen and carbon monoxide, compositions (including reactors, fermenters, bioreactors, devices) for using them, and methods for making and using them. In alternative embodiments, the genetically or recombinantly engineered nitrogen-fixing, nitrogenase expressing bacteria include nitrogen-fixing diazotrophs such as nitrogen-fixing bacteria of the family Pseudomonadaceae, or the genus Azotobacter, including Azotobacter vinelandii, for the whole cell synthesis of hydrocarbons and generating hydrogen and carbon monoxide.

### Continuous Hydrocarbon Formation by CO/Air Cycling

In alternative embodiments, provided are systems (such as bioreactors and devices) and methods for whole cell production of hydrocarbons. We have demonstrated that by cycling the gas atmosphere of a Azotobacter vinelandii culture between carbon monoxide (CO) and air, the A. vinelandii can be alleviated from the inhibitory effects of CO and achieve continuous hydrocarbon production. Using this exemplary system and method, we observed a 20-fold increase in hydrocarbon yield per batch of bacteria.

This exemplary embodiment enhances whole cell hydrocarbon production and streamlines the process since the need to re-culture the bioreactor is greatly reduced. Re-establishment of bacteria cultures can be a procedure involving multiple steps and requiring a few days. In alternative embodiments, by using the systems (such as bioreactors) and methods as provided herein, a culture can be utilized for at least 20 hydrocarbon generation cycles before there is any decreased activity.

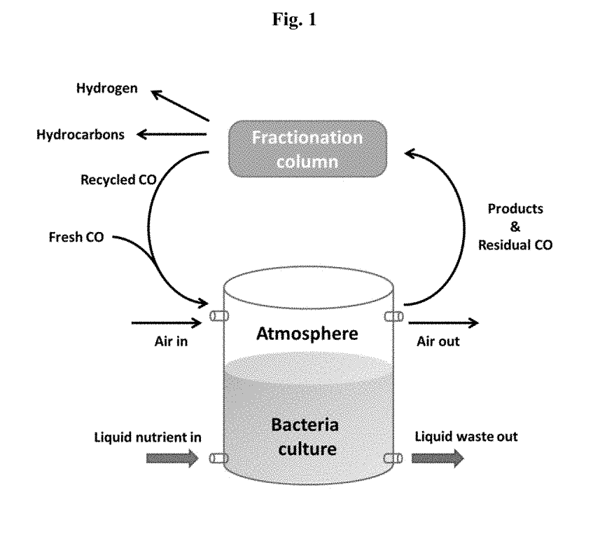

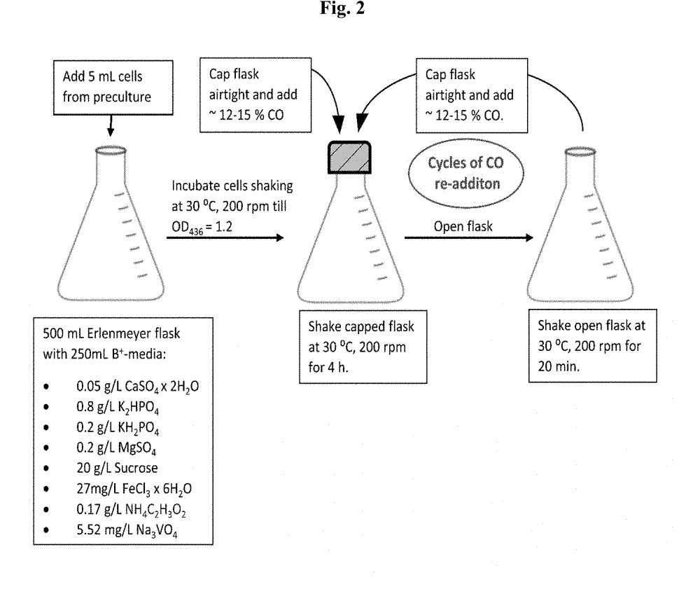

In addition, exemplary embodiments provide a continuous process where hydrocarbons are perpetually produced can be devised, given a suitable system that can replenish the nutrients in the media and remove waste products. Furthermore, each atmospheric cycling step can be integrated with the hydrocarbon harvest process, so that the products that are formed in each cycle can be trapped before returning the unreacted CO back to the reactor (see schematically illustrated exemplary processes of FIG. 1 and FIG. 2).

In alternative embodiments, provided are systems (such as bioreactors and devices) and methods wherein the bacteria used for the production of hydrocarbons are of the family Pseudomonadaceae, or the genus Azotobacter, including Azotobacter vinelandii, such as A. vinelandii, strain YM68A. A. vinelandii with the following genetic modifications were used to practice this exemplary embodiment: (a) deletion of genes encoding the molybdenum transporter and (b) addition of an affinity-tag to the vanadium nitrogenase; and provided are bacteria of the family Pseudomonadaceae, or the genus Azotobacter, including Azotobacter vinelandii, having one or both of these mutations to practice the systems (such as bioreactors) and methods as provided herein.

In one exemplary embodiment of these systems (such as bioreactors) and methods, e.g., as illustrated in FIG. 1, a specific protocol was used as illustrated in FIG. 2. The Azotobacter vinelandii strain YM68A expressing a vanadium nitrogenase and having deletion of genes encoding the molybdenum transporter was grown in 500 ml flasks containing 250 ml Burke's minimal medium supplemented with 2 mM ammonium and 30 μM Na₃VO₄ (designated B⁺-media, see scheme of FIG. 2) at 30° C. (shaker speed: 200 rpm). After reaching an OD₄₃₆=1.2 the flasks are capped airtight, and 12% to 15% CO are added. Following this, the cells are incubated at 30° C. (shaker speed: 200 rpm) for 4 hours (h). After 4 hours the formed hydrocarbons are quantified by a GC-FID. Subsequently the flasks were opened for 20 minutes to allow air exchange. Subsequently, flasks are re-capped and the cycle of hydrocarbon formation is re-started by the addition of 12-15% CO. This cycling can be repeated up to about 20 times without significant reduction of the yield of hydrocarbon formation.

### Hydrogen Production Under CO

In alternative embodiments, provided are systems (such as bioreactors and devices) and methods for whole cell production of hydrogen, as schematically illustrated in FIG. 1. We observed that our Azotobacter vinelandii strain containing vanadium nitrogenase (YM68A) produces a substantial amount of hydrogen under CO. Hydrogen production is elevated by 2.5-fold in the presence of CO compared to that in pure air. Additionally, under 15% CO, hydrogen generation by YM68A is 20-fold higher compared to the molybdenum nitrogenase-containing control strain DJ1141. Overall, the yield of hydrogen is even greater than that of hydrocarbons.

This finding demonstrates that the exemplary systems (such as bioreactors) and methods provided herein are very effective systems for the production of two different fuel-related products, i.e. hydrocarbons and hydrogen, in a single process. In alternative embodiments, hydrogen harvested in this exemplary process are either stored and merchandised or directly fed back into the system as feedstock if further hydrogenation of given products is desired. In alternative embodiments, produced H2 and remaining CO is packaged together as valuable synthesis gas (syngas, H₂+CO) for a renewable source of the typically coal-derived gas mixture.

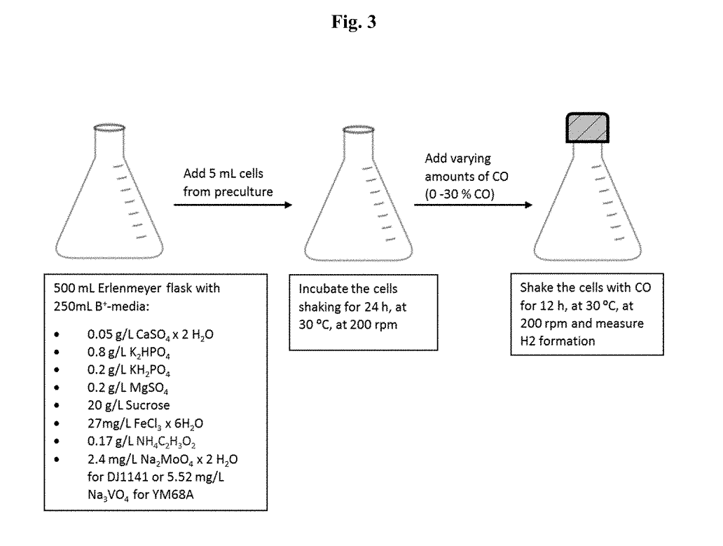

In one exemplary embodiment of these systems (such as bioreactors) and methods, e.g., as illustrated in FIG. 1, a specific protocol was used as illustrated in FIG. 3. Azotobacter vinelandii YM68A expressing V-nitrogenase and having the following genetic modifications was used in this exemplary embodiment: (a) deletion of genes encoding the molybdenum transporter and (b) addition of an affinity-tag to the vanadium nitrogenase. Azotobacter vinelandii DJ1141 expressing an affinity-tagged version of molybdenum nitrogenase (Mo-nitrogenase) was used as a control strain.

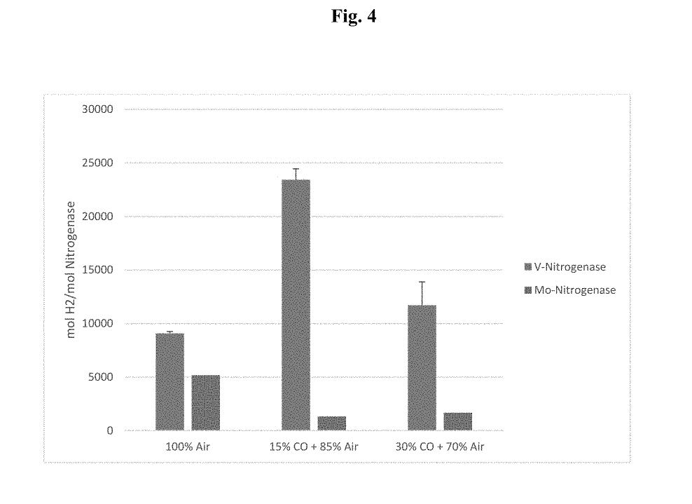

The A. vinelandii strain YM68A (V-nitrogenase) and DJ1141 (Mo-nitrogenase) were grown at 30 ° C. in 500 ml flaks containing 250 ml Burke's minimal medium (designated B⁺-media, see scheme of FIG. 3) supplemented with 2 mM ammonium acetate (shaker speed: 200 rpm). Equal amounts of Na₂MoO₄ or Na₃VO₄ in Burke's medium are used for cell growth of strain DJ1141 and YM68A, respectively. After 24 h of growth the flasks are capped airtight and the indicated amounts of CO (as graphically illustrated in FIG. 4) are added. Subsequently, the cultures are incubated at 30° C. for 15 h (shaker speed: 200 rpm) and the amount of formed H₂ is quantified by a gas chromatograph (GC), e.g., a GC-TCD (a GC with a thermal conductivity detector).

As the data graphically illustrated in FIG. 4, which shows the mol H₂/mol nitrogenase as a function of 100% air, 15% CO and 85% air and 30% CO and 70% air (with V-nitrogenase as the left bar of each pair of bars, and V-nitrogenase as the right of each pair of bars): the addition of 15% CO substantially enhances the formation of H₂ by V-nitrogenase (expressed by A A. vinelandii YM68A, the left bar of each pair of bars) to almost 25000 mol Hz/mol nitrogenase. The formed amount of H₂ exceeds that formed by Mo-nitrogenase in the absence of CO (expressed by A. vinelandii DJ1141, red bars) 5-fold. These data demonstrate that (a) CO acts as an activator for H₂-formation by A. vinelandii YM68A in vivo and (b) that large amounts of H₂ can be generated by A. vinelandii YM68A concurrent with the formation of hydrocarbons.

CO Production from CO₂

This discovery permits the conversion of the greenhouse gas CO₂, which is far more ubiquitous than CO, and thereby provides a novel method to reduce carbon emissions. This finding also allows us to design a 2-step process that combines the CO₂ to CO step with the processes illustrated in FIG. 1 that converts CO to hydrocarbons based on the different strains of Azotobacter vinelandii. Thus, in alternative embodiments, the combination of these processes provides a system and methods where CO₂ is recycled into CO, and the produced CO in turn can be used as a feedstock for hydrogen and hydrocarbon fuel production. As such, this exemplary embodiments, the systems and methods provided herein, can both help reduce CO₂ emissions and broaden the spectrum of feedstocks for hydrocarbon production.

This discovery permits the conversion of the greenhouse gas CO₂, which is far more ubiquitous than CO, and thereby provides a novel method to reduce carbon emissions. This finding also allows us to design a 2-step process that combines the CO₂ to CO step with the above described processes that converts CO to hydrocarbons based on the different strains of Azotobacter vinelandii. Thus, in alternative embodiments, the combination of these processes provides a system and methods where CO₂ is recycled into CO, and the produced CO in turn can be used as a feedstock for hydrogen and hydrocarbon fuel production. As such, this exemplary embodiments, the systems and methods provided herein, can both help reduce CO₂ emissions and broaden the spectrum of feedstocks for hydrocarbon production.

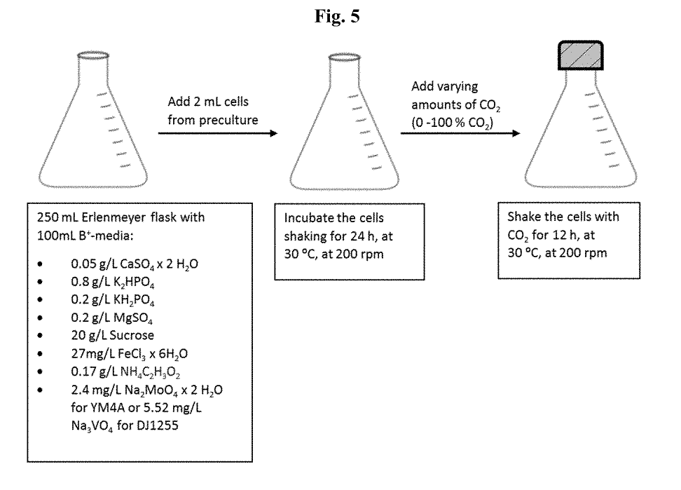

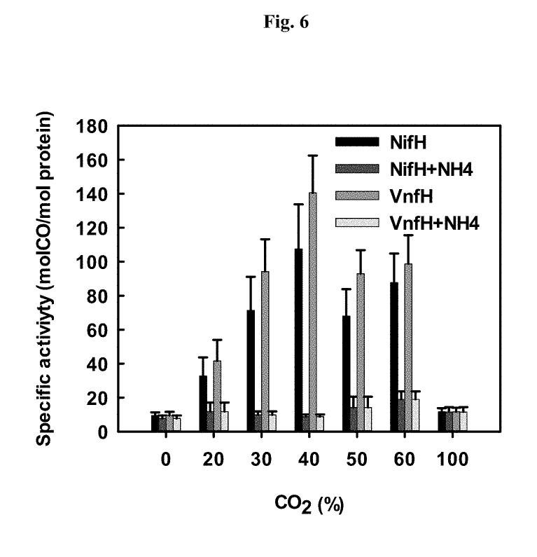

To demonstrate this exemplary embodiment, Azotobacter vinelandii strains with gene deletions that prevent the expression of one or both subunits of the molybdenum-iron or vanadium-iron component of nitrogenase were used; in particular, A. vinelandii strain ΔnifD (for NifH expression of Mo-nitrogenase) and A. vinelandii strain ΔnifDKΔvnfK (for VnfH expression of V-nitrogenase). As schematically illustrated in FIG. 5, both A. vinelandii strains were grown at 30° C. (shaker speed: 200 rpm) in 250 ml flasks containing 100 ml Burke's minimal medium supplemented with 2 mM ammonium acetate (designated B⁺-media; see scheme of FIG. 5). Note that Na₂MoO₄ for the Mo-nitrogenase expressing strain in Burke's medium are replaced by an equal amount of Na₃VO₄ for the V-nitrogenase expressing strain. For the negative control 25 mM ammonium acetate is added to repress the expression of nitrogenase (as graphically illustrated in FIG. 6). After 24 h of growth the flasks are capped airtight and the indicated amount of CO₂ (see FIG. 6) are added. Subsequently, the cells are incubated at 30° C. for 15 h (shaker speed: 200 rpm) and the amount of formed CO is quantified by GC-FID coupled with a methanizer.

FIG. 6 illustrates specific activity in the form of mol CO/mol of protein as a function of 0, 20%, 30%, 40%, 50%, 60%, and 100% CO₂.

The data illustrated in the graph of FIG. 6 shows that the CO formation from CO₂ by genetically modified strains of A. vinelandii expressing NifH of Mo-nitrogenase (most left-hand bar of each four bar set, or the black bars) and VnfH of V-nitrogenase (the second from right of each set of 4 bars, or the green bars). The red (or second from the left of each set of 4 bars) and yellow (or right-hand most bar of each set of 4 bars) bars show control experiments in the presence of ammonia (NH₄) that suppresses the expression of nitrogenase. The data graphically presented in FIG. 6 shows that both strains can generate CO from CO₂ in vivo. These strains could be used as part of a 2-phase technology that converts overall CO₂ to hydrocarbons: 1^{st}—Conversion of CO₂ to CO as provided herein, and 2^{nd}—Conversion of CO to hydrocarbons as described herein.

### Bioreactors, Culture Systems, and Fermenters

In alternative embodiments, provided are culture systems, reactors (bioreactors) and fermenters comprising and comprising use of the exemplary systems and methods provided herein. In alternative embodiments, the culture environments or containers provided herein comprise or are fabricated as culture systems, reactors (bioreactors) and fermenters.

In alternative embodiments, to maximize the efficiency of an industrial process, reactors as provided herein are coupled to existing industrial plants which heavily produce CO or CO₂ as waste; for example, exemplary systems and methods provided herein are coupled to industrial plant exhaust. In alternative embodiments, exemplary systems and methods provided herein are directly tapped into industrial plant exhaust, and exhaust is recycled or reprocessed back into fuel-related products, including hydrocarbons and hydrogen.

To facilitate emission trading, onsite industrial units comprising exemplary systems and methods provided herein are used to reduce a facility's carbon footprint and to spend less money on permits, while at the same time producing valuable products by using their own emissions.

In alternative embodiments, exemplary systems and methods provided herein are scaled according to the required or desired niche and application. For example, exemplary systems and methods provided herein can be run in reactors (bioreactors) fermenters as small 10 liters or up to several thousand liters.

In alternative embodiments, exemplary systems and methods provided herein use an engineered organism whose sole carbon source for growth is CO, making the CO gas both a replacement for sucrose and a feedstock for hydrogen/hydrocarbon production.

In alternative embodiments, the various devices of the invention (e.g., culture systems, bioreactors and fermenters) comprise an inlet configured to provide a carbon-containing compound, particularly “fresh” CO or recycled CO, to an exemplary culture or liquid system in an amount effective to allow a nitrogenase in the nitrogen-fixing, nitrogenase-expressing diazotroph bacteria produce the carbon-carbon bond-comprising product compounds, including hydrocarbons, and hydrogen and CO. In alternative embodiments, various reactors and devices as provided herein further comprise an outlet configured to remove the product of the process, including carbon-carbon bond-comprising product compounds, including hydrocarbons, and hydrogen and CO. In alternative embodiments, separate air in and air out inlets and outlets, respectively, are provided. In alternative embodiments, separate liquid nutrient in and liquid waste out inlets and outlets, respectively, are provided.

In alternative embodiments, the various reactors and devices as provided herein are manufactured or configured to comprise, culture and/or hold a liquid (e.g., a culture media) with exemplary nitrogen-fixing, nitrogenase-expressing diazotroph bacteria as described herein.

In alternative embodiments, the various reactors and devices as provided herein are manufactured or configured to comprise an inlet that permits a carbon-containing compound to be introduced into the liquid in at a rate and/or amount that is effective in providing the nitrogenase with sufficient starting material for the formation of a hydrocarbon, i.e., the carbon-carbon bond-comprising product compound. In alternative embodiments, the various reactors and devices as provided herein are manufactured or configured to comprise an outlet that permits removal of the hydrocarbon product, e.g., the carbon-carbon bond-comprising product compound.

In alternative embodiments, the various reactors and devices as provided herein are manufactured or configured such the nitrogen-fixing, nitrogenase-expressing diazotroph bacteria of the invention are immobilized on a surface, e.g., a semi-solid or a solid surface, which may be conductive. In alternative embodiments, reactors, bioreactors, fermentors and devices as provided herein, or reactors, bioreactors, fermentors and devices used to practice methods and processes as provided herein, can be designed or based on, or can comprise components of, or can be practiced or used, as described by, e.g., U.S. Pat. No. 9,109,193, describing continuous perfusion bioreactor systems; U.S. Pat. No. 9,102,910, describing various bioreactors; U.S. Pat. No. 9,068,215, describing ways to interconnect different bioreactors; U.S. Pat. No. 9,034,640 describing bioreactors with hydrogels and porous membranes; U.S. Pat. No. 9,017,997, describing disposable perfusion bioreactors; U.S. Pat. No. 8,895,291 describing e.g., closed cell expansion systems; U.S. Pat. No. 9,057,044, describing a laminar flow bioreactor with improved laminar flow lines of fluids; U.S. Pat. No. 9,005,959, describing a bioreactor exhaust assembly system; U.S. Pat. No. 8,999,702, describing a disposable bioreactor formed of molded plastic; U.S. Pat. No. 8,889,400 describing e.g., bioreactor systems using gaseous exhausts comprising e.g., carbon monoxide; U.S. Pat. No. 8,865,460 describing e.g., multi-chambered cell co-culture systems; U.S. Pat. No. 8,852,933 describing e.g., flexible, deformable, chambers suitable for seeding and growing cells; U.S. Pat. No. 8,852,925 describing e.g., bioreactors and fermenters comprising three-dimensional matrices, e.g., made of a hydrogel material; U.S. Pat. No. 8,852,923 describing e.g., tissue conditioning bioreactor modules; U.S. Pat. No. 8,835,159 describing e.g., static solid state bioreactors); U.S. Pat. No. 8,828,692 describing e.g., membrane supported bioreactors for conversion of syngas components such as carbon monoxide to liquid products; U.S. Pat. No. 8,518,691 describing e.g., horizontal array bioreactors for conversion of syngas components to liquid products; U.S. Pat. No. 8,222,026 describing e.g., stacked array bioreactors for conversion of syngas components to liquid products; and, U.S. Patent application publications 20150079664, describing hollow fiber bioreactor systems; 20150322397 describing bioreactors with a removable reactor core having internal growth chambers; 20150322396 describing bioreactor arrays with multiple culture vessels with independently controllable inputs is used to culture similar cultures of microorganisms in which at least one parameter differs from other culture vessels in the bioreactor array; 20150315549 describing bioreactors comprising an immobilized enzyme and a heterocyclic compound containing nitrogen and carbon atoms and having 5- or 6-membered ring, which form a reaction field; 20150307828 describing bioreactor vessels for removable connection to a bioreactor module; 20150299636 describing bioreactor apparatus comprising a vessel establishing an interior space environmentally separable from an exterior space outside of said vessel and an agitation system comprising mixing means arranged in an interior space and drive means adapted to rotate the mixing means; 20150290597 describing aeration and mixing devices for disposable flexible bioreactors; 20100105138 describing bioreactors with fluid conveyance systems; and 20140377826 describing bioreactor systems for the biological conversion of CO into desired end products.

A number of embodiments as provided herein have been described. Nevertheless, it can be understood that various modifications may be made without departing from the spirit and scope of the invention. Accordingly, other embodiments are within the scope of the following claims.

1. A whole cell method or system for enzymatically synthesizing a hydrocarbon, a carbon monoxide, a hydrogen or a hydrocarbon, carbon monoxide and hydrogen, comprising:
(a) providing or having provided a nitrogen-fixing bacteria of the family Pseudomonadaceae, wherein optionally the nitrogen-fixing bacteria of the family Pseudomonadaceae is of the genus Azotobacter, optionally an Azotobacter vinelandii,
wherein the bacteria are genetically or recombinantly engineered to lack, substantially lack or have decreased molybdenum transporter activity, optionally by deletion of a molybdenum transporter gene or by inhibition of molybdenum transporter expression, optionally by DNA or RNA targeting by a CRISPR-Cas9 system,
and optionally the bacteria are genetically or recombinantly engineered to: express an exogenous nitrogenase, wherein optionally the exogenous nitrogenase is an exogenous a vanadium nitrogenase; express more endogenous nitrogenase, wherein optionally the endogenous nitrogenase is an endogenous vanadium nitrogenase; and/or have increased nitrogenase activity, wherein optionally the increased nitrogenase activity is an increased vanadium nitrogenase activity,
(b) providing a culture environment or a container for the nitrogen-fixing bacteria of (a), wherein the culture environment or container comprises:
a culture fluid or media for growing or culturing the nitrogen-fixing bacteria,
a liquid input to the culture fluid or media for inputting liquid nutrient and a liquid outlet for outputting liquid waste; and
a gas or air input for inputting gas and a gas or culture atmosphere outlet for outputting or releasing gas;
(c) providing a gas and a hydrocarbon separation device operatively linked to the culture environment or container, wherein the gas input and the gas outlet of the culture environment or container are operably connected to the gas and the hydrocarbon separation device, wherein gas output of the culture environment or container passes through the gas outlet to the gas and hydrocarbon separation device, which separates out or (substantially removes hydrogen and/or hydrocarbons from the gas output of the culture environment or container, and the gas output of the culture environment or container from which hydrogen and/or hydrocarbons are at least substantially removed are returned to the culture environment or container through the gas input for the culture environment or container;
(d) culturing or incubating the nitrogen-fixing bacteria in the culture environment or container under conditions wherein the nitrogen-fixing bacteria generate hydrocarbons, CO and/or hydrogen, and inputting to the culture fluid or media a liquid nutrient, and outputting from the culture fluid or media a liquid waste, and inputting to the culture fluid or media a gas or air mixture comprising CO and air, and outputting gas from the culture fluid or media to the gas and hydrocarbon separation device.

2. The whole cell method or system of claim 1, wherein the suitable culture fluid or media comprises a Burke's minimal medium or equivalent supplemented with 2 mM ammonium or equivalent and 30 μM Na3VO4 or equivalent.

3. The whole cell method or system of claim 1, wherein the gas and hydrocarbon separation device comprises more than one device or apparatus, or comprises a gas chromatograph (GC) or a GC-TCD (a GC with a thermal conductivity detector), or a GC-FID (a GC with flame ionization detector) optionally with methanizer, or equivalents.

4. The whole cell method or system of claim 1, wherein the hydrocarbons produced or generated by the nitrogen-fixing bacteria and separated by the gas and hydrocarbon separation device comprise propane (C3H8), ethane (C2H6), ethylene (C2H4) or any C2 to C10 hydrocarbon, optionally comprising alkanes and alkenes.

5. The whole cell method or system of claim 1, wherein the hydrocarbons, hydrogen and/or CO produced or generated by the nitrogen-fixing bacteria and separated by the gas and hydrocarbon separation device are separated and separately saved or harvested, and optionally all or part of the CO is recycled back to the culture environment or a container.

6. The whole cell method or system of claim 1, wherein the hydrogen and CO produced or generated by the nitrogen-fixing bacteria and separated by the gas and hydrocarbon separation device are harvested and packaged together to produce a syngas.

7. The whole cell method or system of claim 1, wherein the hydrogen produced or generated by the nitrogen-fixing bacteria and separated by the gas and hydrocarbon separation device is recycled back to the culture environment or a container, optionally for hydrogenation of hydrocarbons generated by the nitrogen-fixing bacteria.

8. The whole cell method or system of claim 1, wherein the A. vinelandii comprises an A. vinelandii strain YM68A.

9. The whole cell method or system of claim 1, comprising a system as illustrated in FIG. 1, FIG. 2, FIG. 3 or FIG. 5.

10. A whole cell method or system for enzymatically converting a carbon dioxide to a carbon monoxide and/or a hydrocarbon, comprising:
(a) providing or having provided a nitrogen-fixing bacteria of the family Pseudomonadaceae, wherein optionally the nitrogen-fixing bacteria of the family Pseudomonadaceae is of the genus Azotobacter, optionally an Azotobacter vinelandii,
wherein the bacteria are genetically or recombinantly engineered to: lack, substantially lack or have decreased activity in one or both subunits of the molybdenum-iron (MoFe) or vanadium-iron (VFe) component of nitrogenase (NifD and NifK for MoFe component, or VnfD and VnfK for VFe component, respectively),
and either: permit the expression of an iron protein component of a nitrogenase (NifH for Mo-nitrogenase, VnfH for V-nitrogenase), augment expression of an iron protein component of a nitrogenase, and/or genetically or recombinantly engineer an enzymatic activity comprising an iron protein component of a nitrogenase;
(b) providing a culture environment or a container for the nitrogen-fixing bacteria of (a), wherein the culture environment or container comprises:
a culture fluid or media for growing or culturing the nitrogen-fixing bacteria,
a liquid input to the culture fluid or media for inputting liquid nutrient and a liquid outlet for outputting liquid waste; and
a gas or air input for inputting gas and a gas or culture atmosphere outlet for outputting or releasing gas;
(c) culturing or incubating the nitrogen-fixing bacteria in the culture environment or container under conditions wherein the nitrogen-fixing bacteria generate hydrocarbons and/or CO, and inputting to the culture fluid or media a liquid nutrient, and outputting from the culture fluid or media a liquid waste, and inputting to the culture fluid or media a gas or air mixture comprising carbon dioxide, and outputting gas from the culture fluid or media.

11. The whole cell method or system of claim 10, further comprising operably linking with a method wherein the carbon monoxide or hydrocarbon generated by the method of claim 10 is inputted or recycled into the culture environment or a container of the method,
wherein the method comprises:
(a) providing or having provided a nitrogen-fixing bacteria of the family Pseudomonadaceae, wherein optionally the nitrogen-fixing bacteria of the family Pseudomonadaceae is of the genus Azotobacter, optionally an Azotobacter vinelandii,
wherein the bacteria are genetically or recombinantly engineered to lack, substantially lack or have decreased molybdenum transporter activity, optionally by deletion of a molybdenum transporter gene or by inhibition of molybdenum transporter expression, optionally by DNA or RNA targeting by a CRISPR-Cas9 system,
and optionally the bacteria are genetically or recombinantly engineered to: express an exogenous nitrogenase, wherein optionally the exogenous nitrogenase is an exogenous a vanadium nitrogenase; express more endogenous nitrogenase, wherein optionally the endogenous nitrogenase is an endogenous vanadium nitrogenase; and/or have increased nitrogenase activity, wherein optionally the increased nitrogenase activity is an increased vanadium nitrogenase activity,
(b) providing a culture environment or a container for the nitrogen-fixing bacteria of (a), wherein the culture environment or container comprises:
a culture fluid or media for growing or culturing the nitrogen-fixing bacteria,
a liquid input to the culture fluid or media for inputting liquid nutrient and a liquid outlet for outputting liquid waste; and
a gas or air input for inputting gas and a gas or culture atmosphere outlet for outputting or releasing gas;
(c) providing a gas and a hydrocarbon separation device operatively linked to the culture environment or container, wherein the gas input and the gas outlet of the culture environment or container are operably connected to the gas and the hydrocarbon separation device, wherein pas output of the culture environment or container passes through the gas outlet to the gas and hydrocarbon separation device, which separates out or substantially removes hydrogen and/or hydrocarbons from the gas output of the culture environment or container, and the gas output of the culture environment or container from which hydrogen and/or hydrocarbons are at least substantially removed are returned to the culture environment or container through the gas input for the culture environment or container;
and optionally carbon monoxide (CO) is also separated out or (optionally substantially removed from the gas output of the culture environment or container by the gas and hydrocarbon separation device and recycled back to the culture environment or container through the gas input for the culture environment or container,
and optionally the CO-comprising gas output of the gas and hydrocarbon separation device is mixed with additional CO before inputting to the culture environment or container, and optionally sufficient additional CO is added to the CO-comprising gas output of the gas and hydrocarbon separation device such that a relatively stable amount of CO is recycled into or passed into the culture environment or container,
and optionally the amount of CO recycled or passed back into the culture environment or container is in the form of a CO gas-air mixture comprising between about 5% to 35% CO, between about 12% to 15% CO, or between about 10% to 17% CO, or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or 31% CO,
and optionally the amount of CO recycled or passed back into the culture environment or container is regulated or maintained by a value and a valve actuator or equivalent,
wherein optionally the valve actuator or equivalent is operably linked to a CO detection device in the culture environment or container and an operating system such that the amount of CO passed into the culture environment or container by the value and value actuator maintains the culture environment or container gas environment at between about 5% to 35% CO, between about 12% to 15% CO, or between about 10% to 17% CO, or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or 31% CO; and
(d) culturing or incubating the nitrogen-fixing bacteria in the culture environment or container under conditions wherein the nitrogen-fixing bacteria generate hydrocarbons, CO and/or hydrogen, and inputting to the culture fluid or media a liquid nutrient, and outputting from the culture fluid or media a liquid waste, and inputting to the culture fluid or media a gas or air mixture comprising CO and air, and outputting gas from the culture fluid or media to the gas and hydrocarbon separation device.

12. The whole cell method or system of claim 10, wherein the gas or air mixture comprising carbon dioxide inputted to the culture fluid or media a liquid nutrient comprises an air or a gas mixture comprising between about 10% and 90% carbon dioxide.

13. A genetically or recombinantly engineered nitrogen-fixing bacteria of the family Pseudomonadaceae, wherein optionally the nitrogen-fixing bacteria of the family Pseudomonadaceae is of the genus Azotobacter, optionally an Azotobacter vinelandii,
wherein the bacteria are genetically or recombinantly engineered to: lack, substantially lack or have decreased activity in one or both subunits of the molybdenum-iron (MoFe) or vanadium-iron (VFe) component of nitrogenase (NifD and NifK for MoFe component, or VnfD and VnfK for VFe component, respectively),
and either: permit the expression of an iron protein component of a nitrogenase (NifH for Mo-nitrogenase, VnfH for V-nitrogenase), augment expression of an iron protein component of a nitrogenase, and/or genetically or recombinantly engineer an enzymatic activity comprising an iron protein component of a nitrogenase.

14. A device, a bioreactor or a fermenter comprising genetically or recombinantly engineered nitrogen-fixing bacteria as set forth in claim 13.

15. A device, a bioreactor or a fermenter comprising a method or system of claim 1.

16. A device, a bioreactor or a fermenter comprising a method or system of claim 10.

17. The whole cell method or system of claim 1, wherein in step (c): carbon monoxide (CO) is also separated out or substantially removed from the gas output of the culture environment or container by the gas and hydrocarbon separation device and recycled back to the culture environment or container through the gas input for the culture environment or container.

18. The whole cell method or system of claim 1, wherein in step (c): the CO-comprising gas output of the gas and hydrocarbon separation device is mixed with additional CO before inputting to the culture environment or container, and optionally sufficient additional CO is added to the CO-comprising gas output of the gas and hydrocarbon separation device such that a relatively stable amount of CO is recycled into or passed into the culture environment or container.

19. The whole cell method or system of claim 1, wherein in step (c): the amount of CO recycled or passed back into the culture environment or container is in the form of a CO gas-air mixture comprising between about 5% to 35% CO, between about 12% to 15% CO, or between about 10% to 17% CO, or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or 31% CO.

20. The whole cell method or system of claim 1, wherein in step (c): the amount of CO recycled or passed back into the culture environment or container is regulated or maintained by a value and a valve actuator or equivalent,
wherein optionally the valve actuator or equivalent is operably linked to a CO detection device in the culture environment or container and an operating system such that the amount of CO passed into the culture environment or container by the value and value actuator maintains the culture environment or container gas environment at between about 5% to 35% CO, between about 12% to 15% CO, or between about 10% to 17% CO, or about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or 31% CO.
